(19) United States
(12) Patent Application Publication
(10) Pub. No.: US 2024/0200120 A1
(43) Pub. Date: Jun. 20, 2024

(54) METHODS AND KITS FOR MEASURING PROTEASE ACTIVITY IN FEED

(71) Applicants: DSM IP ASSETS B.V., Heerelen (NL); NOVOZYMES A/S, Bagsvaerd (DK)

(72) Inventors: Claire Véronique CHATELLE, Kaiseraugst (CH); Otto Martin LEHMANN, Kaiseraugst (CH)

(21) Appl. No.: 17/909,867

(22) PCT Filed: Mar. 3, 2021
(86) PCT No.: PCT/EP2021/055314

§ 371 Date: Sep. 7, 2022

(30) Foreign Application Priority Data
Mar. 10, 2020 (EP) 20162187.7

(51) Int. Cl. C12Q 1/37

(52) U.S. Cl. CPC C12Q 1/37

(57) ABSTRACT

The present invention provides a method and a kit for measuring protease activity in a feed sample. The method and the kit avoids the influence of feed inhibitors present in a feed sample and thus provides a stable and accurate method for measuring protease activity in the feed sample with high sensitivity.

### TECHNICAL FIELD

The present invention relates to methods and kits for measuring protease activity in feed.

### BACKGROUND OF THE INVENTION

Proteases are commonly added to animal feed in order to increase the protein digestibility of the feed. In some cases, the protease is added to the feed prior to the pelleting process, which involves heating the feed mixture to high temperatures. In other instances, the protease enzyme is sprayed onto and/or mixed into the feed. In either way, it is often desirable to measure the amount of protease activity in the feed product to make sure that the protease has been in fact added, and the protease has been added in the correct quantity, and the protease survives the pelleting and/or mixing process.

However, there are several difficulties in measuring the protease activities in feed. Firstly, the added protease may be adsorbed into the components of the feed, making it difficult to be extracted. Secondly, the presence of protease inhibitors in some feed has negatively impact and make it difficult to accurately qualify the protease activity. Thirdly, the inclusion level of protease is quite low in the feed, so a highly sensitive method must be built to measure the protease activity therein. Due to these difficulties, existing methods often involve special equipment, complicated procedures or specialized knowledge, or have reduced accuracy and precision.

Therefore, there is a need for simple, highly sensitive methods to detect protease activity in feed that can be performed easily and without the need of complex equipment.

### SUMMARY OF THE INVENTION

The present invention provides a method for measuring protease activity in a feed sample. The method comprises incubating a feed extract containing the protease that is being measured with a protease substrate in a reaction buffer containing sodium dodecyl sulfate (SDS) and measuring the protease activity by inspecting the color change in the reaction mixture.

The present invention also provides a kit for measuring protease activity in a feed sample. The kit comprises an extract solution for preparing the feed extract, a protease substrate, and a reaction buffer containing SDS. In the embodiment of quantitative measurement, the kit according to the present invention further comprises one or more standard enzyme solutions.

### BRIEF DESCRIPTION OF THE DRAWINGS

FIG. 1 are pictures showing the difference in color change between a tube containing 150,000 U/Kg protease (left tube) and a tube without protease (right tube);

FIG. 2 is a coordinate graph showing the protease activity assay results performed with different concentrations of SDS in the reaction buffer;

FIG. 3 is the standard curve prepared by using reference standard solutions of protease ProAct360;

FIG. 4 is a coordinate graph showing the protease activity assay results performed on the protease ProAct;

FIG. 5 is a coordinate graph showing the protease activity assay results performed on the protease AxtraPro; and

FIG. 6 is a coordinate graph showing the protease activity assay results performed on a protease in feed extracted with or without SDS.

### DETAILED DESCRIPTION OF THE INVENTION

The present invention provides a method for measuring protease activity in a feed sample, comprising the following steps:
a) extracting the feed sample with an extracting solution to obtain a feed extract;
b) mixing an aliquot of the feed extract with a protease substrate and a reaction buffer to form a reaction mixture, wherein the protease substrate is a polypeptide coupled to a chromophore and the reaction buffer comprises sodium dodecyl sulfate (SDS);
c) incubating the reaction mixture under conditions such that the chromophore is released from the protease substrate and leads a color change in the reaction mixture; and
d) measuring the protease activity by inspecting the color change in the reaction mixture.

In the present invention, the term “feed sample” can be replaced by “feed”, “food”, “premix” or “food sample”, and refers to any sample containing a protease to be measured. The feed sample according to the present invention may include one or more components of an animal feed. Non-limiting examples of components of an animal feed include: corn or a component of corn such as corn meal, corn fiber, corn hulls, corn DDGS (distiller's dried grain with solubles), silage, ground corn, corn germ, corn gluten, corn oil, and any other portion of a corn plant; soy or a component of soy such as soy oil, soy meal, soy hulls, soy silage, ground soy, and any other portion of a soy plant; wheat or any component of wheat such as wheat meal, wheat fiber, wheat hulls, wheat chaff, ground wheat, wheat germ, and any other portion of a wheat plant; barley or a component of a barley plant; glycerol; lecithin; rumen protected fats; molasses; grasses such as orchard grass and fescue; fish meal, meat & bone meal; feather meal; and poultry byproduct meal; and alfalfa and/or clover used for silage or hay, and various combinations of any of the feed ingredients set forth herein, or other feed ingredients generally known in the art. As it will be recognized in the art, a feed composition may further be supplemented with amino acids, vitamins, minerals, and other feed additives such as other types of enzymes, organic acids, essential oils, probiotics, prebiotics, antioxidants, pigments, anti-caking agents, and the like.

According to the present invention, the feed sample may be in any suitable form known in the field of the animal feed and may be a wet or dry component. For example, the feed sample may be in a form selected from the group consisting of a complete feed, a feed supplement, a feed additive, a premix, a top-dress, a tub, a mineral, a meal, a block, a pellet, a mash, a liquid supplement, a drench, a bolus, a treat, and combinations of any thereof. Additionally, the feed sample may optionally be ground before extracted with the extracting solution according to the present invention.

The feed of the present invention may be formulated for administration to any animal. The animal includes but is not limited to human, food animals such as poultry (e.g., chickens, including broilers, layers, and breeders, ducks, game hens, geese, guinea fowl/hens, quail, and turkeys), beef cattle, dairy cattle, veal, pigs, goats, sheep, bison, and fishes; companion animals such as cats, dogs, horses, rabbits, rodents (e.g., mice, rats, hamsters, gerbils, and guinea pigs), hedgehogs, and ferrets; research animals such as rodents, cats, dogs, rabbits, pigs, and non-human primates; and zoo animals such as non-human primates, lions, tigers, bears, elephants, giraffes, and the like.

The feed sample of the present invention may include a protease, which can be measured by the method of the present invention, to improve the digestibility of the feed. The proteases used in the feed sample may be any protease enzyme generally known in the art. Examples of the protease include but are not limited to an aspartic protease, an asparagine protease, a cysteine protease, a glutamic protease, a metalloprotease, a serine protease, and a threonine protease, and combinations thereof. Preferably, the protease is a serine protease. More preferably, the protease is the protease ProAct360 (DSM Nutritional Products Ltd., Switzerland) and/or ProAct (DSM Nutritional Products Ltd., Switzerland) and/or AxtraPro (Dupont, USA).

In the method according to the present invention, the feed sample is firstly subjected to extraction with an extracting solution in the step a).

The extracting solution may be any solution suitable for extracting a protease from the feed sample. One example of the extracting solution is a buffer solution. The buffer solution may be any buffer solution containing one or more of the following buffering reagents: H₂O, sodium chloride (NaCl), glycine, phosphate, acetate, citrate, carbonate, bicarbonate, 4-(2-hydroxyethyl)-1-piperazineethanesulfonic acid (HEPES), and tris(hydroxymethyl)aminomethane (Tris). The extracting solution may or may not contain phosphate. Preferably, the extracting solution is free of phosphate. More preferably, the extracting solution is NaCl solution in distilled water.

The extracting solution may comprise polysorbate surface active agent such as Tween® 20, Tween® 40, Tween® 60 or Tween® 80. Preferably, the extracting solution comprises Tween® 20.

Preferably, the extracting solution comprises Tween® 20 and sodium chloride. More preferably, the extracting solution contains from 1 mM to 500 mM, preferably from 20 mM to 800 mM, more preferably from 50 mM to 500 mM such as 55 mM, 60 mM, 65 mM, 70 mM, 75 mM, 80 mM, 85 mM, 90 mM, 95 mM, 100 mM, 110 mM, 120 mM, 130 mM, 140 mM, 150 mM, 160 mM, 170 mM, 180 mM, 190 mM, 200 mM, 250 mM, 300 mM, 350 mM, 400 mM and 450 mM of sodium chloride, and from 0.001 w/v % to 0.1 w/v %, preferably from 0.005 w/v % to 0.05 w/v % such as 0.006 w/v %. 0.007 w/v %, 0.008 w/v %, 0.009 w/v %, 0.01 w/v %, 0.011 w/v %, 0.012 w/v %, 0.013 w/v %, 0.014 w/v %, 0.015 w/v %, 0.016 w/v %, 0.017 w/v %, 0.018 w/v %, 0.019 w/v %, 0.02 w/v %, 0.025 w/v %, 0.03 w/v %, 0.035 w/v %, 0.04 w/v % or 0.045 w/v % of Tween® 20.

The amount of the feed sample used for the extraction of the step a) may vary. In general, the amount of the feed sample may be from about 1 gram to about 100 grams, such as about 2 gram, 5 grams, 10 grams, 15 grams, 20 grams, 25 grams, 30 grams, 35 grams, 40 grams, 45 grams, 50 grams, 55 grams, 60 grams, 70 grams, 80 grams, 90 grams or 95 grams, or more. Preferably, from about 10 grams to about 60 grams of the feed sample is used. More preferably, about 20 grams to about 50 grams of feed is used. Further preferably, about 20 grams or about 50 grams of feed is used for the extraction.

The extracting solution may be used in any amount suitable for the purpose of extraction. Preferably, the extracting solution is used in the step a) in an amount of from about 1 mL to about 100 mL, preferably from about 5 mL to about 50 mL, more preferably from about 10 ml to 20 mL, per 1 gram of the feed used in the step.

The extraction of the step a) may be carried out at room temperature to obtain a feed extract. Optionally, the feed extract may be further operated, such as by centrifugation, before it is putted into the next step.

In the step b) of the method according to the invention, the feed extract or its aliquot obtained from the step a) is mixed with a protease substrate and a reaction buffer to form a reaction mixture.

In the step b), the protease substrate may be any polypeptide coupled to a chromophore which can be cleaved by the protease that is being measured and thus release the chromophore to lead a color change in the reaction mixture.

In the present invention, the term “polypeptide” is used in its broadest sense and may include peptides, polypeptides, and proteins, as well as peptides, polypeptides, and proteins that contain one or more non-natural amino acids or any other chemical modification that allows the polypeptide to function as a substrate for a protease enzyme whose activity is being measured. The polypeptide may be a naturally occurring polypeptide, such as casein, collagen, gelatin, albumin, globin, and the like. Alternatively, the polypeptide may be a synthetic peptide or polypeptide.

In the present invention, the term “chromophore” is a chemical group which may provide visible change in color if it is released into a solution. Non-limiting examples of suitable chromophores include but are not limited to Erioglaucine, Reactive Black 5, Reactive Blue 21, Reactive Orange 78, Reactive Yellow 15, Reactive Blue 19, Reactive Blue 4, Reactive Red 11, Reactive Yellow 86, Reactive Blue 163, Reactive Red 180, mono- and di-halogentriazine dyes such as mono- and di-fluorotriazine dyes, mono- and di-chlorotriazine dyes, mono-(m′-carboxypyridinium) triazines, dyes in the PROCION® line of dyes, the CIBACRON™ line of coal tar colors, 2,4,5-trihalogenopyriminidines, 2,3-dihaloquinoxalines, N-hydroxysulfosuccinimidyl (sulfo-NHS) ester functionalized dyes, N-hydroxysuccinimidyl (NHS) functionalized dyes, vinyl sulfone dyes such as the REMAZOL® line of coal tar dyestuffs including REMAZOL® Blue, azo dyes such as Sudan I, Sudan II, Sudan III, Sudan IV, Sudan Black, Disperse Orange, Disperse Red, Oil Red O, Trypan Blue, Congo Red, ß-carotene, p-nitroanilide (pNA), sulfonyl chloride dyes such as lissamine, rhodamine, and dabsyl chloride, tetrafluorophenyl ester functionalized dyes, isothiocyanate functionalized dyes, and iodoacetyl functionalized dyes and other dyes that are structurally equivalent to the dyes listed herein.

The protease substrate may be insoluble in the reaction mixture. Non-limiting examples of insoluble substrates suitable for use in methods of the present invention include hide-Remazol Brilliant Blue R, azo collagen, Azurine cross-linked casein, gelatin-Remazol Brilliant Blue, casein-Remazol Brilliant Blue, and collagen-Remazol Brilliant Blue. In an exemplary embodiment, an insoluble substrate is casein-Remazol Brilliant Blue.

Alternatively, the protease substrate may be soluble in the reaction mixture. Non-limiting examples of suitable soluble substrates include N-succinyl-Ala-Ala-Pro-Phe-pNA, N-succinyl-Ala-Ala-Pro-Leu-pNA, N-succinyl-Ala-Ala-Pro-Arg-pNA, N-methylsulfonyl-D-Phe-Gly-Arg-pNA, and o-aminobenzoyl-AGSRGAGQ-(2,3-dinitrophenyl-ethylene diamine). Preferably, the substrate is N-succinyl-Ala-Ala-Pro-Phe-pNA.

In the step b), the reaction buffer may be any buffer suitable for protease cleavage reaction know in the art. The reaction buffer of the present invention may comprise one or more buffering reagents selected from the group consisting of Tris, Tris HCl, 3-{[tris(hydroxymethyl)methyl]amino}propanesulfonic acid (TAPS), N,N-bis(2-hydroxyethyl)glycine (Bicine), N-tris(hydroxymethyl)methylglycine (tricine), 3-[N-Tris(hydroxymethyl)methylamino]-2-hydroxypropanesulfonic acid (TAPSO), HEPES, 2-{[tris(hydroxymethyl)methyl]amino}ethanesulfonic acid (TES), 3-(N-morpholino)propanesulfonic acid (MOPS), piperazine-N,N′-bis(2-ethanesulfonic acid) (PIPES), dimethylarsinic acid (cacodylate), saline sodium citrate (SSC), 2-(N-morpholino)ethanesulfonic acid (MES), 2(R)-2-(methylamino)succinic acid (succinic acid), borate, phosphate, acetate, glycine, magnesium or calcium carbonate, and bicarbonate. Preferably, the reaction buffer of the present invention comprises glycine as buffering reagent. More preferably, the reaction buffer of the present invention is a glycine-sodium hydroxide buffer.

The pH of a reaction buffer may vary. For example, the reaction buffer in the method of the present invention may be adjusted to a pH of from about 6.0 to about 12.0, such as 6.0, 6.5, 7.0, 7.5, 8.0, 8.5, 9.0, 9.5, 10.0, 10.5, 11.0, 11.5, and about 12.0. Preferably, the reaction buffer has a pH of from about 7.0 to about 11.0, such as 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9 or about 11.0. More preferably, the reaction buffer has a pH of from about 7.5 to about 10.0, such as 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or about 10.0. The most preferably, the reaction buffer has a pH of about 9.0.

The concentration of the buffering agent in the reaction buffer is typically sufficient to maintain a desired pH range and as a result, may vary. The concentration of the buffering agent in the reaction buffer for example may be from about 20 mM to about 200 mM, such as 20, 30, 40, 50, 60, 70, 80, 90, 100, 110, 120, 130, 140, 150, 160, 170, 180, 190 and 200 mM. Preferably, the concentration of the buffering agent in the reaction buffer is from about 50 mM to 150 mM, such as 50, 60, 70, 80, 90, 100, 110, 120, 130, 140 and 150 mM. More preferably, the concentration of the buffering agent in the reaction buffer is about from 60 mM to 120 mM, such as 60, 65, 70, 75, 80, 85, 90, 95, 100, 105, 110, 115 and 120 mM. Further preferably, the concentration of the buffering agent in the reaction buffer is from about 90 mM to about 110 mM, such as 90, 91, 92, 93, 94, 95, 96, 97, 98, 99, 100, 101, 102, 103, 104, 105, 106, 107, 108, 109 and 110 mM. The most preferably, the concentration of buffering agent in the reaction buffer is about 100 mM.

The reaction buffer according to the present invention may contain one or more surface acting agent. The surface acting agent may be a polysorbate surface active agent such as Tween® 20, Tween® 40, Tween® 60, or Tween® 80. More preferably, the surface acting agent is Tween® 20. The reaction buffer may contain the surface acting agent in an amount of from 0.001 w/v % to 0.1 w/v %, preferably from 0.005 w/v % to 0.05 w/v % such as 0.006 w/v %. 0.007 w/v %, 0.008 w/v %, 0.009 w/v %, 0.01 w/v %, 0.011 w/v %, 0.012 w/v %, 0.013 w/v %, 0.014 w/v %, 0.015 w/v %, 0.016 w/v %, 0.017 w/v %, 0.018 w/v %, 0.019 w/v %, 0.02 w/v %, 0.025 w/v %, 0.03 w/v %, 0.035 w/v %, 0.04 w/v % or 0.045 w/v % of Tween® 20.

The reaction buffer according to the present invention may contain sodium dodecyl sulfate (SDS). The concentration of SDS in the reaction buffer may be in the range of from about 0.0005 w/v % to about 0.5 w/v %, such as 0.0005 w/v %, 0.0006 w/v %, 0.0007 w/v %, 0.0008 w/v %, 0.0009 w/v %, 0.001 w/v %, 0.002 w/v %, 0.003 w/v %, 0.004 w/v %, 0.005 w/v %, 0.006 w/v %, 0.007 w/v %, 0.008 w/v %, 0.009 w/v %, 0.01 w/v %, 0.02 w/v %, 0.03 w/v %, 0.04 w/v %, 0.05 w/v %, 0.06 w/v %, 0.07 w/v %, 0.08 w/v %, 0.09 w/v %, 0.1 w/v %, 0.2 w/v %, 0.3 w/v %, 0.4 w/v %, and 0.5 w/v %. Preferably, the concentration of SDS in the reaction buffer may be in the range of from about 0.005 w/v % to about 0.5 w/v %, such as 0.005 w/v %, 0.015 w/v %, 0.025 w/v %, 0.035 w/v %, 0.045 w/v %, 0.055 w/v %, 0.065 w/v %, 0.075 w/v %, 0.085 w/v %, 0.095 w/v %, 0.15 w/v %, 0.25 w/v %, 0.35 w/v % and 0.45 w/v %. More preferably, the concentration of SDS in the reaction buffer may be in the range of from about 0.01 w/v % to about 0.45 w/v %, such as 0.01 w/v %, 0.02 w/v %, 0.03 w/v %, 0.04 w/v %, 0.05 w/v %, 0.06 w/v %, 0.07 w/v %, 0.08 w/v %, 0.09 w/v %, 0.1 w/v %, 0.2 w/v %, 0.3 w/v %, 0.4 w/v %, 0.41 w/v %, 0.42 w/v %, 0.43 w/v %, 0.44 w/v % and 0.45 w/v %.

The reaction buffers may further comprise salts such as sodium chloride (NaCl) or potassium chloride (KCl), reducing agents such as dithiothreitol (DTT), β-mercaptoethanol (BME), and tris(2-carboxyethyl)phosphine (TCEP), bulking agents such as dextran sulfate, polyethylene glycol (PEG), an anti-foaming agent, enzymatic inhibitors, and/or tetraethylene glycol, and/or others.

Preferably, the reaction buffer contains glycine as buffering reagent, one or more surface acting agent such as Tween® 20, SDS and/or salts such as sodium chloride (NaCl). More preferably, the reaction buffer contains glycine in an amount of from about 50 mM to about 150 mM, such as 50, 55, 60, 65, 70, 75, 80, 85, 90, 95, 100, 105, 110, 115, 120, 125, 130, 135, 140, 145 and 150 mM; Tween® 20 in an amount of from 0.005 w/v % to 0.05 w/v % such as 0.006 w/v %, 0.0065 w/v %, 0.007 w/v %, 0.0075 w/v %, 0.008 w/v %, 0.0085 w/v %, 0.009 w/v %, 0.0095 w/v %, 0.01 w/v %, 0.011 w/v %, 0.012 w/v %, 0.013 w/v %, 0.014 w/v %, 0.015 w/v %, 0.016 w/v %, 0.017 w/v %, 0.018 w/v %, 0.019 w/v %, 0.02 w/v %, 0.025 w/v %, 0.03 w/v %, 0.035 w/v %, 0.04 w/v % or 0.045 w/v %; SDS in an amount of from 0.005 w/v % to about 0.1 w/v %, such as 0.005 w/v %, 0.006 w/v %, 0.007 w/v %, 0.008 w/v %, 0.009 w/v %, 0.01 w/v %, 0.015 w/v %, 0.02 w/v %, 0.025 w/v %, 0.03 w/v %, 0.035 w/v %, 0.04 w/v %, 0.045 w/v %, 0.05 w/v %, 0.055 w/v %, 0.06 w/v %, 0.065 w/v %, 0.07 w/v %, 0.075 w/v %, 0.08 w/v %, 0.085 w/v %, 0.09 w/v %, 0.095 w/v % and 0.1 w/v %; and/or NaCl in an amount of from 100 mM to 1000 mM such as 100 mM, 150 mM, 200 mM, 250 mM, 300 mM, 350 mM, 400 mM, 450 mM, 500 mM, 550 mM, 600 mM, 6500 mM, 700 mM, 750 mM, 800 mM, 850 mM, 900 mM and 1000 mM.

In the step b), the reaction buffer may be added in any amount to make the chromophore coupled to the protease substrate is cleaved smoothly by the protease that is being measured in the reaction mixture. Preferably, the reaction buffer is added in an amount from 50 mL to 500 mL, more preferably from 60 mL to 400 mL, further preferably from 70 mL to 200 mL such as 80, 90, 100, 110, 120, 130, 140, 150, 160, 170, 180, 190, and 200 mL, per 1 mL of the feed extract added into the reaction mixture.

In the step b), the protease substrate should be added in a sufficient amount to make the protease that is being measured cleaves and releases the chromophore as much as possible from the protease substrate in the reaction mixture. Preferably, the protease substrate is added in an amount to make the reaction mixture contains from 0.5 mM to 20 mM, more preferably from 1 mM to 15 mM, further preferably from 2 mM to 10 mM such as 2, 3, 4, 5, 6, 7, 8, 9 or 10 mM of the protease substrate.

In the step b), the feed extract or the aliquot thereof may be mixed with the protease substrate and the reaction buffer directly, or after dilution. Preferably, the feed extracted is diluted 1:1-100 (v/v), more preferably 1:5-80 (v/v), more preferably 1:50 (v/v) with the reaction buffer before mixture.

Once the reaction mixture is prepared as step b), the method of the present invention further involves a step c) incubating the reaction mixture under conditions that allows the protease substrate to be cleaved by the protease that is being measured and releases the chromophore to lead a color change in the reaction mixture.

As understood by any person skilled in the art, the conditions and time required for incubation of the reaction mixture would vary depending on the feed sample, the concentration and nature of the protease in the feed sample, and the identity of the protease substrate used in preparing a reaction mixture. In general, the reaction mixture may be incubated at a temperature ranging from about ambient temperature to about 100° C. For instance, the reaction mixture may be incubated at a temperature of about 15, 20, 25, 30, 35, 40, 45, 50, 55, 60, 65, 70, 75, 80, 85, 90, 95, or about 100° C. Typically, the reaction mixture is incubated until a sufficient amount of the chromophore is developed to allow for determination of protease activity. For instance, the duration of incubation may range from about 1 minute to about 10 hours or more. Preferably, the incubation is carried out at 65° C. for 1 hour.

The next step of the method of the present invention comprises a step d) measuring the protease activity by inspecting the color change in the reaction mixture.

In the step d) of the present invention, the protease activity may be measured qualitatively through visual inspection of the color change generated by the chromophore released in the reaction mixture.

In the present invention, the term “qualitatively” refers to a simple determination of the presence or absence of protease in the feed sample. As such, a method of the present invention may be used to determine if a protease has been added to the feed sample and/or if the protease activity has survived the feed preparation process. Generally, when protease activity is measured qualitatively, protease activity is measured through visual inspection of the color change generated by the chromophore released in the reaction mixture.

When protease activity is measured qualitatively, the method of the present invention may further be optimized to determine the presence of a minimum amount of protease activity in a feed sample. The minimum amount of protease activity in a feed sample may be at least about 0.03 mg enzyme/Kg feed, such as 0.03, 0.031, 0.032, 0.033, 0.034, 0.035, 0.036, 0.037, 0.038, 0.039, 0.04, 0.042, 0.045, 0.046, 0.048, 0.05, 0.055, 0.06, 0.065, 0.07, 0.08, 0.09 and 0.1 mg enzyme/Kg feed.

Alternatively, the protease activity in the step d) of the present invention may be measured quantitatively through spectrophotometric detection of the color change generated by the chromophore released in the reaction mixture. The inventors surprisingly discovered that the method of the present invention is especially valuable for quantitative measure of protease activity in a feed sample.

In the present invention, the term “quantitatively” refers to the accurate determination of the amount of protease activity in a feed sample. For instance, a method of the present invention may be used to determine the absolute amount of protease activity present in a feed sample or how much of the protease activity has survived the feed preparation process.

In one embodiment, the protease activity in the step d) of the present invention may be measured quantitatively through comparison of the color change generated by the chromophore in the reaction mixture to the color change in a standard curve of one or more of the feed samples comprising a known amount of the protease that is being measured. It is well known for any person skilled in the art on how to prepare the standard curve and how to calculate the protease activity according to the comparison to the standard curve (see Harris, T. K. et al., Guide to Protein Purification, 2nd Edition, Methods in Enzymology 2009 463:57-71).

When protease activity is measured quantitatively, the method of the present invention is suitable to determine the protease activity over a wide range of protease concentrations. Preferably, the method of the present invention may be suitable for detect protease activity of at least about 0.2 mg enzyme/Kg feed, such as 0.2, 0.21, 0.22, 0.23, 0.24, 0.25, 0.26, 0.27, 0.28, 0.29 and 0.3 mg enzyme/Kg feed.

The method of the present invention avoids the influence of feed inhibitors present in a feed sample and thus provides a stable and accurate method for measuring protease activity in the feed sample with high sensitivity. Especially it is suitable for quantitatively measuring protease activity in the feed sample. It also avoids complex equipment, so it is simple, cheap and suitable for automation.

The present invention also provides a kit for measuring protease activity in a feed sample. The kit of the present invention may contain: i) a extracting solution as described above, ii) a protease substrate which is a polypeptide coupled to a chromophore as described above; iii) a reaction buffer comprising as described above, (iv) an instruction manual for the following steps: a) extracting a feed sample with the extracting solution to obtain a feed extract, b) mixing an aliquot of the feed extract with the protease substrate and the reaction buffer to form a reaction mixture, c) incubating the reaction mixture under conditions such that the cleavage of the protease substrate leads a color change in the reaction mixture; and d) measuring the protease activity by inspecting the color change in the reaction mixture.

In one embodiment of the invention, the kit may be used to measure protease activity in a feed sample qualitatively.

In another embodiment of the invention, the kit of the present invention is used to measure protease activity in a feed sample quantitatively. In such an embodiment, the kit may further contain v) one or more standard enzyme solutions for preparing a standard curve.

The kits of the present invention can avoid the use of expensive equipment and can be used easily at the site of feed/food processing.

The following examples are included to further illustrate the present invention.

### EXAMPLES

### Example 1

The following experiment was performed to visually assess the presence of a protease ProAct360 (DSM Nutritional Products Ltd., Switzerland) in a feed sample.

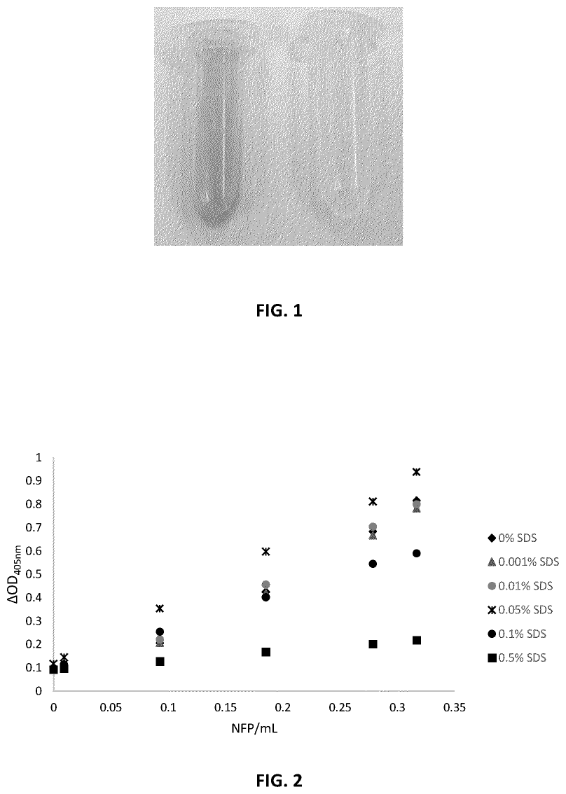

50 g of a feed sample containing protease ProAct360 (150,000 U/Kg) were extracted with 500 ml extraction solution (100 mM NaCl, 0.01 w/v % Tween® 20) for 1 hour. 2 mL of the extract was centrifuged and diluted 1/50 in glycine-sodium hydroxide buffer (100 mM Glycine, 500 mM NaCl, 0.01 w/v % Tween® 20, 0.05 w/v % SDS, adjust the pH with 5 M NaOH solution to pH 9). 100 μl of the diluted extract were mixed with 100 μl of the 4 mM substrate Suc-Ala-Ala-Pro-Phe-pNA (Bachem AG, Switzerland) solution in a tube. The solution was incubated for 1 hour at 65° C. The reaction was stopped by adding 0.2 mL ethanol. A negative control containing no protease was performed in parallel in a separate tube. FIG. 1 indicated the color change of the two tubes. The presence of a yellow coloration in the left tube, sign of the presence of a protease activity, was visually assessed, while the right tube containing no protease has no color change.

### Example 2

The following experiment was performed to develop and optimize a protease assay for measuring protease activity in feed. In this example, different SDS concentrations were evaluated for their suitability to denature inhibitors while maintaining the protease activity.

20 g of blank feed samples were spiked with different amounts (0, 5,000, 50,000, 100,000, 140,000, 150,000 NFP/Kg) of protease ProAct360 (DSM Nutritional Products Ltd., Switzerland) and extracted using 200 ml extraction solution (100 mM NaCl, 0.01 w/v % Tween® 20) respectively. 2 mL of the extracts was centrifuged and diluted 1/50 in glycine-sodium hydroxide buffer (100 mM Glycine, 500 mM NaCl, 0.01 w/v % Tween® 20, adjust the pH with 5 M NaOH solution to pH 9) containing different concentrations of SDS (0, 0.001, 0.01, 0.05, 0.1 and 0.5% (w/v)). 100 μL of the diluted extracts were mixed with 100 μl of the 4 mM substrate Suc-Ala-Ala-Pro-Phe-pNA (Bachem AG, Switzerland) solution in separate tubes. The solutions were incubated for 1 hour at 65° C. The reactions were stopped by adding 0.2 mL EtOH and the OD_{405 nm} was measured on each tube with a spectrophotometer.

FIG. 2 indicated the proportional relationship between the protease concentrations with the values of OD_{405 nm} in the presence of different concentrations of SDS. There was an increase in OD_{405 nm} with increased enzyme concentration. The highest increase was observed for a concentration of 0.05 w/v % SDS, allowing for a higher assay sensitivity. Lower concentration of SDS seemed to be insufficient to denature the inhibitors, while higher concentrations may denature the tested protease.

### Example 3

Different feed samples (mash and pelleted feed) were analyzed using the described method, the in-feed protease activity was quantified using a standard curve with a reference standard enzyme solution.

### Preparation of Standard Curve

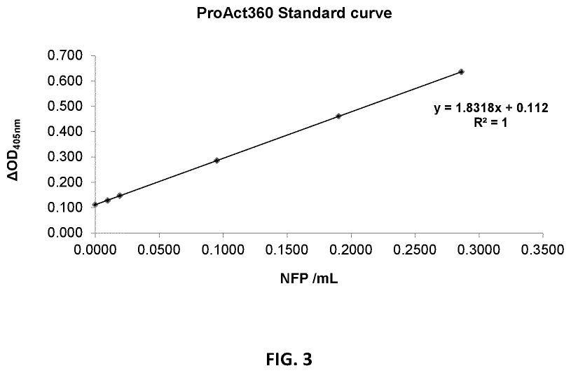

20 g of blank feed samples were spiked with different amounts (0, 16.5, 15, 10, 5, 0.5 NFP/mL) of protease ProAct360 (DSM Nutritional Products Ltd., Switzerland) and extracted using 200 ml extraction solution (100 mM NaCl, 0.01 w/v % Tween® 20) respectively. 2 mL of the extracts were centrifuged and diluted 1/50 in glycine-sodium hydroxide buffer (100 mM Glycine, 500 mM NaCl, 0.01 w/v % Tween® 20, 0.05 w/v % SDS, adjust the pH with 5 M NaOH solution to pH 9). 100 μL of the diluted extracts were mixed with 100 μL of the 4 mM substrate Suc-Ala-Ala-Pro-Phe-pNA (Bachem AG, Switzerland) solution in separate tubes. The solutions were incubated for 1 hour at 65° C. The reaction was stopped by adding 0.2 mL EtOH and the OD_{405 nm} was measured on each tube with a spectrophotometer. A standard curve (FIG. 3) was provided by plotting the OD_{405 nm} values of the samples with different amounts of protease ProAct360 on the y-axis and the protease activity on the x-axis.

### Measuring Protease Activities in Different Feed Samples

50 g feed samples were extracted with 500 mL extraction solution (100 mM NaCl, 0.01 w/v % Tween® 20) for 1 hour. 2 mL of the extracts were centrifuged and diluted 1/50 in glycine-sodium hydroxide buffer (100 mM Glycine, 500 mM NaCl, 0.01 w/v % Tween® 20, 0.05 w/v % SDS, adjust the pH with 5 M NaOH solution to pH 9). 100 μL of the diluted extract were mixed with 100 μL of the 4 mM substrate Suc-Ala-Ala-Pro-Phe-pNA (Bachem AG, Switzerland) solution in separate tubes. The solutions were incubated for 1 hour at 65° C. The reaction was stopped by adding 0.2 mL EtOH and the OD_{405 nm} was measured on each tube with a spectrophotometer. The in-feed activity of each sample was calculated based on the standard curve prepared as above. The results are shown in Table 1 as below:

TABLE 1
Measured protease activity in different
feed samples (mash and pellets)

| | ΔOD₄₀₅ₙₘ | Protease Activity (NFP/Kg) | | |
|---|---|---|---|---|
| Samples | Value | Calculated Value | Mean | RSDᵣ, % |
| Pellet 1 | 0.139 | 9,260 | 9,773 | 7.42% |
| | 0.142 | 10,286 | | |
| Mash 1 | 0.144 | 10,879 | 11,414 | 6.62% |
| | 0.148 | 11,948 | | |
| Pellet 2 | 0.282 | 46,477 | 47,385 | 2.71% |
| | 0.290 | 48,292 | | |
| Mash 2 | 0.320 | 56,774 | 57,848 | 2.63% |
| | 0.329 | 58,922 | | |
| Pellet 3 | 0.687 | 156,617 | 160,250 | 3.21% |
| | 0.716 | 163,883 | | |
| Mash 3 | 0.755 | 174,990 | 167,660 | 6.18% |
| | 0.699 | 160,329 | | |

### Example 4

The suitability of the method of the present invention for the measurement of different proteases in feed was evaluated. Two commonly used in-feed protease products ProAct (DSM Nutritional Products Ltd., Switzerland) and AxtraPro (Dupont, USA) were tested.

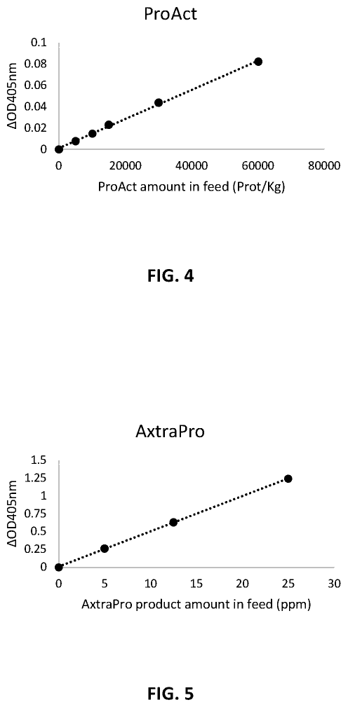

20 g blank feed were spiked with different amounts of the proteases (ProAct: 0, 5,000, 10,000, 15,000, 30,000, 60,000 Prot/Kg feed; and AxtraPro: 0, 5, 12.5, and 25 ppm) respectively. Each of the spiked samples was extracted with 200 ml extraction solution (100 mM NaCl, 0.01 w/v % Tween® 20). 2 mL of the obtained extracts was centrifuged and diluted 1/50 in glycine-sodium hydroxide buffer (100 mM Glycine, 500 mM NaCl, 0.01 w/v % Tween® 20, 0.05 w/v % SDS, adjust the pH with 5 M NaOH solution to pH 9). 100 μL of the diluted extract were mixed with 100 μl of the 4 mM substrate Suc-Ala-Ala-Pro-Phe-pNA (Bachem AG, Switzerland) solution in separate tubes. The solutions were incubated for 1 hour at 65° C. The reactions were stopped by adding 0.2 mL EtOH and the OD_{405 nm} was measured on each tube with a spectrophotometer. For every protease, a coordinate graph was made by plotting the OD_{405 nm} values of the samples with different amounts of the protease on the y-axis and the protease activity on the x-axis (FIG. 4 and FIG. 5). As shown in FIG. 4 and FIG. 5, the increase OD_{405 nm} values was proportional to the increased enzyme activity, demonstrating the suitability of the method for the measurement of the different proteases in feed samples.

### Example 5

The following experiment was performed to compare the method of the present invention with the invention wherein the SDS is added to the extraction solution.

20 g of blank feed samples were spiked with different amount (0, 5,000, 50,000, 100,000, 140,000, 150,000 NFP/Kg) of protease ProAct360 (DSM Nutritional Products Ltd., Switzerland), respectively. Each of the spiked sample was extracted with 200 ml extraction solution (100 mM NaCl, 0.01 w/v % Tween® 20) containing SDS (0% or 0.01% (w/v)). 2 ml of the extracts were centrifuged and diluted 1/50 in glycine-sodium hydroxide buffer (100 mM Glycine, 500 mM NaCl, 0.01 w/v % Tween® 20, adjust the pH with 5 M NaOH solution to pH 9) containing SDS (0% or 0.05% (w/v)). 100 μl of the diluted extract were mixed with 100 μl of the 4 mM substrate Suc-Ala-Ala-Pro-Phe-pNA (Bachem AG, Switzerland) solution in separate tubes. The solutions were incubated for 1 hour at 65° C. The reaction was stopped by adding 0.2 mL EtOH and the OD_{405 nm} was measured on each tube with a spectrophotometer.

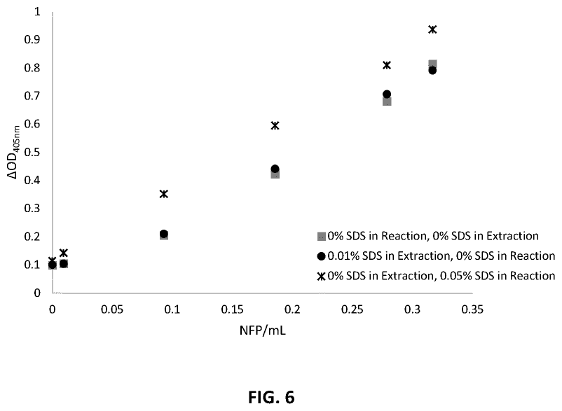

A coordinate graph was made by plotting the OD_{405 nm} values of the samples with different amounts of the proteases on the y-axis and the protease activity on the x-axis (FIG. 6). As shown in FIG. 6, there was an increase in OD_{405 nm} with increased enzyme activities. The highest increase was observed using the method containing SDS in reaction solution, so the reaction happening with 0.05% SDS allows a higher assay sensitivity.

1. A method for measuring protease activity in a feed sample, comprising the following steps:
a) extracting the feed sample with an extracting solution to obtain a feed extract;
b) mixing an aliquot of the feed extract with a protease substrate and a reaction buffer to form a reaction mixture, wherein the protease substrate is a polypeptide coupled to a chromophore, and the reaction buffer comprises sodium dodecyl sulfate (SDS);
c) incubating the reaction mixture under conditions such that the chromophore is released from the protease substrate and leads a color change in the reaction mixture; and
d) measuring the protease activity by inspecting the color change in the reaction mixture.

2. The method of claim 1, wherein the feed is any forms of feed for animals including but not limited to human, food animals such as poultry (e.g., chickens, including broilers, layers, and breeders, ducks, game hens, geese, guinea fowl/hens, quail, and turkeys), beef cattle, dairy cattle, veal, pigs, goats, sheep, bison, and fishes; companion animals such as cats, dogs, horses, rabbits, rodents (e.g., mice, rats, hamsters, gerbils, and guinea pigs), hedgehogs, and ferrets; research animals such as rodents, cats, dogs, rabbits, pigs, and non-human primates; and zoo animals such as non-human primates, lions, tigers, bears, elephants, giraffes, and the like.

3. The method of claim 1, wherein the protease is any one or more selected from the group consisting of an aspartic protease, an asparagine protease, a cysteine protease, a glutamic protease, a metalloprotease, a serine protease and a threonine protease, and preferably it is the protease ProAct and/or ProAct360 and/or AxtraPro.

4. The method of claim 1, wherein the extracting solution is a buffer solution containing one or more of the buffering reagents selected from the group consisting of H₂O, sodium chloride (NaCl), glycine, phosphate, acetate, citrate, carbonate, bicarbonate, 4-(2-hydroxyethyl)-1-piperazineethanesulfonic acid (HEPES), tris(hydroxymethyl)aminomethane (Tris).

5. The method of claim 1, wherein the extracting solution comprises polysorbate surface active agent such as Tween® 20, Tween® 40, Tween® 60 or Tween® 80.

6. The method of claim 1, wherein the extracting solution contains from 1 mM to 500 mM, preferably from 20 nM to 800 mM, more preferably from 50 mM to 500 mM such as 55 mM, 60 mM, 65 mM, 70 mM, 75 mM, 80 mM, 85 mM, 90 mM, 95 mM, 100 mM, 110 mM, 120 mM, 130 mM, 140 mM, 150 mM, 160 mM, 170 mM, 180 mM, 190 mM, 200 mM, 250 mM, 300 mM, 350 mM, 400 mM and 450 mM of sodium chloride, and from 0.001 w/v % to 0.1 w/v %, preferably from 0.005 w/v % to 0.05 w/v % such as 0.006 w/v %, 0.007 w/v %, 0.008 w/v %, 0.009 w/v %, 0.01 w/v %, 0.011 w/v %, 0.012 w/v %, 0.013 w/v %, 0.014 w/v %, 0.015 w/v %, 0.016 w/v %, 0.017 w/v %, 0.018 w/v %, 0.019 w/v %, 0.02 w/v %, 0.025 w/v %, 0.03 w/v %, 0.035 w/v %, 0.04 w/v % or 0.045 w/v % of Tween® 20.

7. The method of claim 1, wherein the protease substrate is selected from the group consisting of N-succinyl-Ala-Ala-Pro-Phe-pNA, N-succinyl-Ala-Ala-Pro-Leu-pNA, N-succinyl-Ala-Ala-Pro-Arg-pNA, N-methylsulfonyl-D-Phe-Gly-Arg-pNA, and o-aminobenzoyl-AGSRGAGQ-(2,3-dinitrophenyl-ethylene diamine.

8. The method of claim 1, wherein the reaction buffer comprises one or more buffering reagents selected from the group consisting of tris(hydroxymethyl)aminomethane (Tris), Tris HCl, 3-{[tris(hydroxymethyl)methyl]amino}propanesulfonic acid (TAPS), N,N-bis(2-hydroxyethyl)glycine (Bicine), N-tris(hydroxymethyl)methylglycine (tricine), 3-[N-Tris(hydroxymethyl)methylamino]-2-hydroxypropanesulfonic acid (TAPSO), 4-(2-hydroxyethyl)-1-piperazineethanesulfonic acid (HEPES), 2-{[tris(hydroxymethyl)methyl]amino}ethanesulfonic acid (TES), 3-(N-morpholino)propanesulfonic acid (MOPS), piperazine-N,N′-bis(2-ethanesulfonic acid) (PIPES), dimethylarsinic acid (cacodylate), saline sodium citrate (SSC), 2-(N-morpholino)ethanesulfonic acid (MES), 2(R)-2-(methylamino)succinic acid (succinic acid), borate, phosphate, acetate, glycine, magnesium or calcium carbonate, and bicarbonate.

9. The method of claim 1, wherein the concentration of SDS in the reaction buffer is in the range of from about 0.001 w/v % to about 0.5 w/v %, preferably from about 0.005 w/v % to about 0.1 w/v %, more preferably from about 0.01 w/v % to about 0.08 w/v %, such as 0.01 w/v %, 0.02 w/v %, 0.03 w/v %, 0.04 w/v %, 0.05 w/v %, 0.06 w/v %, 0.07 w/v %, and 0.08 w/v %.

10. The method of claim 1, wherein the reaction buffer comprises one or more surface acting agent such as Tween® 20, Tween® 40, Tween® 60, or Tween® 80.

11. The method of claim 1, wherein the reaction buffer comprises salts such as sodium chloride (NaCl) or potassium chloride (KCl), reducing agents such as dithiothreitol (DTT), β-mercaptoethanol (BME), and tris(2-carboxyethyl)phosphine (TCEP), bulking agents such as dextran sulfate, polyethylene glycol (PEG), an anti-foaming agent, enzymatic inhibitors, and/or tetraethylene glycol, and/or others.

12. The method of claim 1, wherein the reaction buffer contains glycine as buffering reagent, one or more surface acting agent such as Tween® 20, SDS and/or salts such as sodium chloride (NaCl).

13. The method of claim 1, wherein the reaction mixture is incubated at a temperature of about 15, 20, 25, 30, 35, 40, 45, 50, 55, 60, 65, 70, 75, 80, 85, 90, 95, or about 100° C.

14. The method of claim 1, wherein the protease activity is measured qualitatively or quantitively.

15. A kit for measuring protease activity in a feed sample, comprising: i) a extracting solution, ii) a protease substrate which is a polypeptide coupled to a chromophore; iii) a reaction buffer comprising sodium dodecyl sulfate (SDS), (iv) an instruction manual for the following steps: a) extracting the feed sample with the extracting solution to obtain a feed extract, b) mixing an aliquot of the feed extract with the protease substrate and the reaction buffer to form a reaction mixture, c) incubating the reaction mixture under conditions such that the cleavage of the protease substrate leads a color change in the reaction mixture; and d) measuring the protease activity by inspecting the color change in the reaction mixture.

16. The kit of claim 15, which further comprises v) one or more standard enzyme solutions for preparing a standard curve.
